(19) United States
(12) Patent Application Publication
(10) Pub. No.: US 2025/0027017 A1
(43) Pub. Date: Jan. 23, 2025

(54) DEVICE FOR OBSERVING MICROORGANISMS AND METHOD OF OBSERVING MICROORGANISMS

(71) Applicant: NIPPON TELEGRAPH AND TELEPHONE CORPORATION, Tokyo (JP)

(72) Inventors: Manami ITO, Musashino-shi (JP); Sosuke IMAMURA, Musashino-shi (JP); Kazuhiro TAKAYA, Musashino-shi (JP); Kaori TAKAYANAGI, Musashino-shi (JP); Nobuaki ENDO, Musashino-shi (JP)

(73) Assignee: NIPPON TELEGRAPH AND TELEPHONE CORPORATION, Tokyo (JP)

(21) Appl. No.: 18/713,716

(22) PCT Filed: Dec. 8, 2021
(86) PCT No.: PCT/JP2021/045182

§ 371 Date: May 28, 2024

(51) Int. Cl. C12M 1/00; C12M 1/12; C12Q 1/02

(52) U.S. Cl. CPC C12M 23/34; C12M 23/04; C12M 23/40; C12Q 1/02

(57) ABSTRACT

A device for observing microorganisms includes a laminate of a first board and a second board. The second board is provided with a recess, a first groove, and a second groove on a surface facing the first board. The first board or the second board is provided with an inlet hole, an outlet hole, and a reagent feed hole. The recess is connected to the first groove and constitutes a culturing section for culturing microorganisms. The first groove has one end connected to the inlet hole and the other end connected to the outlet hole. The second groove has one end connected to the reagent feed hole and the other end connected to the first groove. At least one of the first board and the second board has light permeability in an observation area corresponding to a part of the first groove.

### TECHNICAL FIELD

The present invention relates to a device for observing microorganisms and method of observing microorganisms.

### BACKGROUND ART

Microfluidic devices for culturing cells such as microorganisms are known. A microfluidic device has a size equivalent to, for example, that of a glass slide, and the inside of the device is provided with a channel having a width of several tens of micrometers and a cell-culturing space, and the front surface of the device is provided with an inlet hole and an outlet hole constituting ends of the channel.

When culturing microorganisms with a microfluidic device, an amount of a product produced by the microorganisms (hereinafter also referred to as a microorganism-producing product) may be measured to observe the activity of the microorganisms. In this case, it is necessary to add a reagent to a microorganism-culturing space in order to measure the amount of the microorganism-producing product, but there is concern that the reagent may affect the growth of the microorganisms.

### CITATION LIST

### Non Patent Literature

Non Patent Literature 1: Tarryn E. Miller et al. 2020. “Light-Powered CO2 Fixation in a Chloroplast Mimic with Natural and Synthetic Parts”. Science 368 (6491): 649-654.

### SUMMARY OF INVENTION

### Technical Problem

An object of the present invention is to provide a technique related to a microfluidic device capable of detecting or quantifying a microorganism-producing product without affecting the growth of microorganisms.

### Solution to Problem

According to one aspect of the invention,
there is provided a device for observing microorganisms, including a laminate of a first board and a second board,
in which the second board is provided with a recess, a first groove, and a second groove on a surface facing the first board, and
the first board or the second board is provided with an inlet hole, an outlet hole, and a reagent feed hole,
the recess being connected to the first groove and constituting a culturing section for culturing microorganisms,
the first groove having one end connected to the inlet hole and the other end connected to the outlet hole,
the second groove having one end connected to the reagent feed hole and the other end connected to the first groove, and
at least one of the first board and the second board having light permeability in an observation area corresponding to a part of the first groove.

According to a second aspect of the present invention,
there is provided a method of observing microorganisms using the device for observing microorganisms according to the first aspect, the method involving:
supplying a culture solution from the inlet hole to the first groove;
culturing the microorganisms in the culturing section in presence of the culture solution;
causing a product produced by the microorganisms to flow out of the culturing section and into the first groove;
supplying a reagent from the reagent feed hole to the second groove; and
observing the culture solution containing the product and the reagent at the observation area.

### Advantageous Effects of Invention

According to the present invention, there is provided a technique related to a microfluidic device capable of detecting or quantifying a microorganism-producing product without affecting the growth of microorganisms.

### BRIEF DESCRIPTION OF DRAWINGS

FIG. 1 is a top view of a microorganism observing device according to an embodiment.

FIG. 2 is a cross-sectional view taken along line II-II of the microorganism observing device illustrated in FIG. 1.

### DESCRIPTION OF EMBODIMENTS

Hereinafter, an embodiment of the present invention will be described. The following embodiment is given for embodying any one of the above aspects. The following matters can be incorporated into each of the above aspects independently or in combination.

### 1. Device for Observing Microorganisms

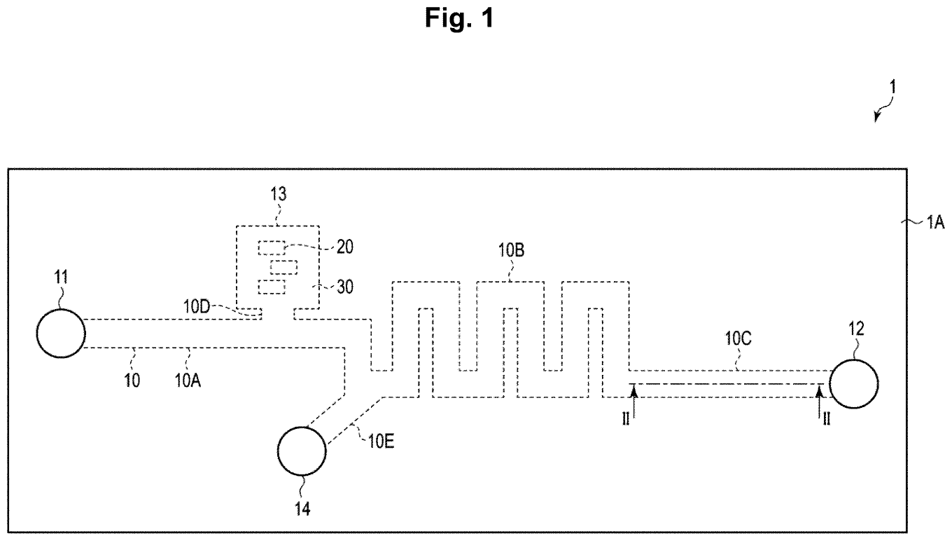

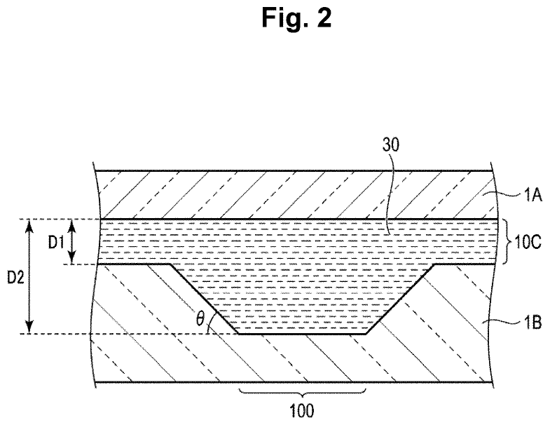

A microorganism observing device according to an embodiment will be described with reference to FIGS. 1 and 2. FIG. 1 is a top view of a microorganism observing device according to an embodiment. FIG. 2 is a cross-sectional view taken along line II-II of the microorganism observing device illustrated in FIG. 1.

As illustrated in FIGS. 1 and 2, the microorganism observing device 1 includes a laminate of a first board 1A and a second board 1B. The first board 1A and the second board 1B each have light permeability in an observation area 100 corresponding to an observation section of a third channel 10C. Note that both of the first board 1A and the second board 1B do not necessarily have light permeability in the observation area 100 corresponding to the observation section of the third channel 10C as long as at least one of them has light permeability in the observation area 100 corresponding to the observation section of the third channel 10C. Alternatively, the whole body of at least one of the first board 1A and the second board 1B may have light permeability. The “light permeability” herein is a property that enables observation of a culture solution containing a microorganism-producing product and a reagent. The “light permeability” is, for example, visible light permeability.

Examples of a material used in the first board 1A and the second board 1B include polydimethylsiloxane (PDMS) and glass. The microorganism observing device 1 as a whole has, for example, a size equivalent to that of a glass slide.

The first board 1A is provided with an inlet hole 11, an outlet hole 12, and a reagent feed hole 14. The inlet hole 11, the outlet hole 12, and the reagent feed hole 14 may be disposed in the second board 1B. The inlet hole 11 is used for feeding a culture solution or other kinds of liquids into a main channel 10 (to be described) from the outside of the microorganism observing device 1. The outlet hole 12 is used for discharging the culture solution or other kinds of liquids from the main channel 10 to the outside of the microorganism observing device 1. The reagent feed hole 14 is used for feeding a reagent from the outside of the microorganism observing device 1 into a first sub channel 10E (to be described).

A surface of the second board 1B facing the first board 1A is provided with a recess, a first groove, a second groove, and a third groove. The recess constitutes a culturing section 13 for culturing microorganisms 20. The first groove constitutes the main channel 10. The second groove constitutes the first sub channel 10E. The third groove constitutes a second sub channel 10D.

The culturing section 13 is a chamber for culturing the microorganisms 20. A microorganism suspension containing the microorganisms 20 and a culture solution 30 is stored in the culturing section 13. The microorganisms 20 are, for example, soil microorganisms. The culture solution 30 is a liquid containing a nutrient source for the microorganisms 20. The culturing section 13 is connected to the main channel 10 via the second sub channel 10D. The second sub channel 10D has a function of allowing part of the culture solution 30 supplied from the inlet hole 11 to the main channel 10 to flow into the culturing section 13 and a function of allowing the culture solution containing the product produced by the microorganisms 20 to flow out of the culturing section 13 and into the main channel 10. The second sub channel 10D may be omitted, and the culturing section 13 may be directly connected to the main channel 10.

When viewed from a direction perpendicular to the main surface of the first board 1A, the culturing section 13 has a length of, for example, 10 μm to 1 mm in a width direction of the second sub channel 10D, and a length of, for example, 10 μm to 1 mm in a direction perpendicular to the width direction of the second sub channel 10D. The culturing section 13 has a depth of, for example, 1 μm to 1 mm.

The second sub channel 10D has a width W2 smaller than a dimension W1 of the culturing section 13 in the width direction of the second sub channel 10D. The second sub channel 10D having such a small width W2 makes it difficult to flow the microorganisms 20 out of the culturing section 13 and into the main channel 10.

The width W2 of the second sub channel 10D is, for example, 5 μm to 0.5 mm. A ratio W2/W1 of the width W2 to the width W1 is, for example, 0.2 to 0.5.

The second sub channel 10D has a length of, for example, 2 μm to 0.5 mm. The second sub channel 10D having such a short length makes it easy to flow the culture solution containing the product produced by the microorganisms 20 out of the culturing section 13 and into the main channel 10.

The main channel 10 has one end connected to the inlet hole 11 and the other end connected to the outlet hole 12. The main channel 10 is composed of a first channel 10A, a second channel 10B, and the third channel 10C. The first channel 10A, the second channel 10B, and the third channel 10C are arranged in this order from the inlet hole 11 toward the outlet hole 12.

The first channel 10A is connected to the culturing section 13 via the second sub channel 10D. The first channel 10A is connected to the reagent feed hole 14 via the first sub channel 10E. Accordingly, the culture solution containing the product produced by the microorganisms 20 flows out of the culturing section 13 and into the first channel 10A, and the reagent is supplied from the reagent feed hole 14 to the first channel 10A. The reagent is, for example, a substrate that reacts with the product produced by the microorganisms 20 and produces a color or luminescence. The first channel 10A has a width of, for example, 10 to 800 μm, a depth of, for example, 10 to 1000 μm, and a length of, for example, 1 to 50 mm.

The second channel 10B is located downstream of the first channel 10A and forms a meandering portion. The second channel 10B does not necessarily meander and may be linear. In the second channel 10B, the culture solution containing a microorganism-producing product is mixed with the reagent. The second channel 10B has a width of, for example, 10 to 800 μm, a depth of, for example, 10 to 1000 μm, and a length of, for example, 1 to 50 mm.

The third channel 10C is located downstream of the second channel 10B and includes the observation section. As shown in FIG. 2, in the device, an area where the reagent reacting with the microorganism-producing product and producing a color or luminescence is observed is referred to as the observation area 100. A part of the third channel 10C corresponding to the observation area 100 is referred to as the observation section.

As illustrated in FIG. 2, the observation section has a depth D2 larger than a depth D1 of the third channel 10C other than the observation section. In other words, the depth D2 of the main channel 10 (specifically, the third channel 10C) at a position corresponding to the observation area 100 is larger than the depth D1 of the main channel 10 (specifically, the third channel 10C) at other positions. Forming the observation section to have such a large depth makes it possible to easily observe turbidity of the liquid and diffusion of light within the observation section.

The depth D2 of the observation section is, for example, 10 to 3000 μm. The depth D1 of the third channel 10C other than the observation section is, for example, 10 to 1000 μm. A ratio D2/D1 of the depth D2 to the depth D1 is, for example, 2 to 5. Note that FIG. 2 illustrates a case where the depth D2 of the observation section is larger than the depth D1 of the third channel 10C other than the observation section, but the depth D2 may be the same as the depth D1. The third channel 10C including the observation section has a width of, for example, 10 to 800 μm and a length of, for example, 1 to 50 mm.

As illustrated in FIG. 2, both ends of the observation section are provided with inclined portions. In other words, the depth of the main channel 10 (specifically, the third channel 10C) gradually increases from positions other than the observation area 100 toward the position corresponding to the observation area 100. With the inclined portions, the liquid is prevented from being stagnant in the observation section. The inclined portions have an inclination angle θ of, for example, 45 to 60 degrees. In FIG. 2, the inclined portions (that is, surfaces connecting the bottom surface of the main channel 10 and the bottom surface of the observation section) are flat but may be curved.

As described above, in the microorganism observing device 1 illustrated in FIG. 1, the portion to which the culturing section 13 (specifically, the second sub channel 10D) is connected, the portion to which the first sub channel 10E is connected, the meandering portion, and the observation section are arranged in this order from the inlet hole 11 toward the outlet hole 12.

### Effects

As described above, according to the microorganism observing device 1, part of the culture solution 30 contained in the culturing section 13 is caused to flow out into the main channel 10, and the culture solution 30 flowing out into the main channel 10 is mixed with the reagent fed from the reagent feed hole 14 in the meandering portion, and then the microorganism-producing product contained in the culture solution 30 is detected or quantified at the observation section.

A microfluidic device in the related art requires addition of a reagent to a microorganism-culturing space (culturing section) in order to detect or quantify a microorganism-producing product. In contrast, in the microorganism observing device 1, part of the culture solution 30 contained in the culturing section 13 is caused to flow out into the main channel 10, and the culture solution 30 flowing out is mixed with the reagent, and then the microorganism-producing product contained in the culture solution 30 is detected or quantified. Accordingly, it is possible to observe a condition of microorganisms without affecting the growth of the microorganisms.

In addition, when a substrate that exhibits fluorescence by reacting with a microorganism-producing product is used as the reagent, in the microfluidic device in the related art, the reagent is added to the microorganism-culturing space (culturing section), and the fluorescence in the culturing section fades in color over time, which makes it difficult to observe over a long period of time. In contrast, in the microorganism observing device 1, part of the culture solution 30 contained in the culturing section 13 is caused to flow out into the main channel 10 and used as a sample. For this reason, even after a period of time, it is possible to send a new sample to the observation section, which enables long-duration observation without causing color fading of the fluorescence.

### 2. Method of Observing Microorganisms

According to another aspect, there is provided a method of observing microorganisms using the aforementioned microorganism observing device. According to an embodiment, a method of observing microorganisms using the aforementioned microorganism observing device involves:
supplying the culture solution 30 from the inlet hole 11 to the main channel 10;
culturing the microorganisms 20 in the culturing section 13 in presence of the culture solution 30;
causing a product produced by the microorganisms 20 to flow out of the culturing section 13 and into the main channel 10;
supplying a reagent from the reagent feed hole 14 to the first sub channel 10E; and
observing the culture solution 30 containing the microorganism-producing product and the reagent at the observation area 100.

This method can be implemented, for example, by the following steps. First, a microorganism suspension containing the microorganisms 20 and the culture solution 30 is added to the recess of the second board 1B constituting the culturing section 13. Next, the first board 1A is bonded onto the second board 1B to form the microorganism observing device 1.

After that, the culture solution 30 is supplied from the inlet hole 11 to the main channel 10. Part of the culture solution 30 supplied to the main channel 10 flows into the culturing section 13, and part of the culture solution 30 stored in the culturing section 13 flows out into the main channel 10. Accordingly, the product produced by the microorganisms 20 flows out of the culturing section 13 and into the main channel 10. Herein, only the culture solution 30 may flow out of the culturing section 13 and into the main channel 10, and the microorganisms 20 may not flow out. Alternatively, a part of the microorganisms 20 may flow out together with the culture solution 30 flowing out of the culturing section 13 and into the main channel 10.

In the meantime, the reagent is supplied from the reagent feed hole 14 to the first sub channel 10E. The reagent is typically supplied in the form of a reagent-containing liquid. As described above, a usable example of the reagent is a substrate that reacts with the microorganism-producing product and thereby produces a color or luminescence to the extent that it can be observed under visible light or fluorescence observation. The reagent may be a substrate that produces a color by reacting with a product produced by microorganisms, a substrate that produces chemiluminescence by reacting with a product produced by microorganisms, or a substrate that exhibits fluorescence by reacting with a product produced by microorganisms. For example, Salzmann reagent is applicable as a reagent that reacts with a nitrite salt to produce a color.

The culture solution 30 flowing out into the main channel 10 is mixed with the reagent in the second channel (meandering portion) 10B and stirred sufficiently. The stirring can be performed by alternately switching a forward flow and a reverse flow of the liquid in the main channel 10 to stir the liquid in the meandering portion.

After the stirring, the mixed sample (that is, the culture solution containing a reaction product of the microorganism-producing product and the reagent) in the meandering portion is sent to the observation section of the third channel 10C. The observation section is observed with a microscope, whereby the microorganism-producing product is detected based on the presence or absence of a color or fluorescence. In addition, the color intensity or fluorescence intensity in the observation section may be measured to determine an amount of the microorganism-producing product based on the measured value. On completion of the observation and/or measurement, the mixed sample in the observation section is discharged to the outlet hole 12.

### Effects

A method of observing microorganisms using a microfluidic device in the related art requires addition of a reagent to a microorganism-culturing space (culturing section) in order to detect or quantify a microorganism-producing product. In contrast, in the method of observing microorganisms using the microorganism observing device 1, part of the culture solution 30 contained in the culturing section 13 is caused to flow out into the main channel 10, and the culture solution 30 flowing out is mixed with the reagent, and then the microorganism-producing product contained in the culture solution 30 is detected or quantified. Accordingly, it is possible to observe a condition of microorganisms without affecting the growth of the microorganisms.

In addition, when a substrate that exhibits fluorescence by reacting with a microorganism-producing product is used as the reagent, in the method of observing microorganisms using the microfluidic device in the related art, the reagent is added to the microorganism-culturing space (culturing section), and the fluorescence in the culturing section fades in color over time, which makes it difficult to observe over a long period of time. In contrast, in the method of observing microorganisms using the microorganism observing device 1, part of the culture solution 30 contained in the culturing section 13 is caused to flow out into the main channel 10 and used as a sample. For this reason, even after a period of time, it is possible to send a new sample to the observation section, which enables long-duration observation without causing color fading of the fluorescence.

### REFERENCE SIGNS LIST

1 MICROORGANISM OBSERVING DEVICE
1A FIRST BOARD
1B SECOND BOARD
10 MAIN CHANNEL
10A FIRST CHANNEL
10B SECOND CHANNEL
10C THIRD CHANNEL
10D SECOND SUB CHANNEL
10E FIRST SUB CHANNEL.
100 OBSERVATION AREA
11 INLET HOLE.
12 OUTLET HOLE.
13 CULTURING SECTION
14 REAGENT FEED HOLE
20 MICROORGANISMS
30 CULTURE SOLUTION

1.-8. (canceled)

9. A device for observing microorganisms, comprising a laminate of a first board and a second board,
wherein the second board is provided with a recess, a first groove, and a second groove on a surface facing the first board, and
the first board or the second board is provided with an inlet hole, an outlet hole, and a reagent feed hole,
the recess being connected to the first groove and constituting a culturing section for culturing microorganisms,
the first groove having one end connected to the inlet hole and the other end connected to the outlet hole,
the second groove having one end connected to the reagent feed hole and the other end connected to the first groove, and
at least one of the first board and the second board having light permeability in an observation area corresponding to a part of the first groove.

10. The device for observing microorganisms according to claim 9, wherein the first groove includes a meandering portion.

11. The device for observing microorganisms according to claim 10, wherein a portion of the first groove to which the recess is connected, a portion of the first groove to which the other end of the second groove is connected, the meandering portion, and a portion of the first groove corresponding to the observation area are arranged in this order between the inlet hole and the outlet hole.

12. The device for observing microorganisms according to claim 9, wherein a depth of the first groove at a position corresponding to the observation area is larger than the depth of the first groove at other positions.

13. The device for observing microorganisms according to claim 10, wherein a depth of the first groove at a position corresponding to the observation area is larger than the depth of the first groove at other positions.

14. The device for observing microorganisms according to claim 11, wherein a depth of the first groove at a position corresponding to the observation area is larger than the depth of the first groove at other positions.

15. The device for observing microorganisms according to claim 12, wherein the first groove has a depth gradually increasing from the other position toward the position corresponding to the observation area.

16. The device for observing microorganisms according to claim 13, wherein the first groove has a depth gradually increasing from the other position toward the position corresponding to the observation area.

17. The device for observing microorganisms according to claim 14, wherein the first groove has a depth gradually increasing from the other position toward the position corresponding to the observation area.

18. The device for observing microorganisms according to claim 9, wherein the recess and the first groove are connected via a third groove disposed on the surface of the second board, and the third groove has a width smaller than a dimension of the recess in a width direction of the third groove.

19. The device for observing microorganisms according to claim 10, wherein the recess and the first groove are connected via a third groove disposed on the surface of the second board, and the third groove has a width smaller than a dimension of the recess in a width direction of the third groove.

20. The device for observing microorganisms according to claim 11, wherein the recess and the first groove are connected via a third groove disposed on the surface of the second board, and the third groove has a width smaller than a dimension of the recess in a width direction of the third groove.

21. The device for observing microorganisms according to claim 12, wherein the recess and the first groove are connected via a third groove disposed on the surface of the second board, and the third groove has a width smaller than a dimension of the recess in a width direction of the third groove.

22. The device for observing microorganisms according to claim 13, wherein the recess and the first groove are connected via a third groove disposed on the surface of the second board, and the third groove has a width smaller than a dimension of the recess in a width direction of the third groove.

23. The device for observing microorganisms according to claim 14, wherein the recess and the first groove are connected via a third groove disposed on the surface of the second board, and the third groove has a width smaller than a dimension of the recess in a width direction of the third groove.

24. The device for observing microorganisms according to claim 15, wherein the recess and the first groove are connected via a third groove disposed on the surface of the second board, and the third groove has a width smaller than a dimension of the recess in a width direction of the third groove.

25. The device for observing microorganisms according to claim 16, wherein the recess and the first groove are connected via a third groove disposed on the surface of the second board, and the third groove has a width smaller than a dimension of the recess in a width direction of the third groove.

26. The device for observing microorganisms according to claim 17, wherein the recess and the first groove are connected via a third groove disposed on the surface of the second board, and the third groove has a width smaller than a dimension of the recess in a width direction of the third groove.

27. A method of observing microorganisms using the device for observing microorganisms according to claim 9,
the method comprising:
supplying a culture solution from the inlet hole to the first groove;
culturing the microorganisms in the culturing section in presence of the culture solution;
causing a product produced by the microorganisms to flow out of the culturing section and into the first groove;
supplying a reagent from the reagent feed hole to the second groove; and
observing the culture solution containing the product and the reagent at the observation area.

28. The observation method according to claim 27, wherein the reagent is a substrate that produces a color, chemiluminescence, or fluorescence by reacting with the product, and the observation of the culture solution containing the product and the reagent involves measuring of light intensity.
